(19) United States
(12) Patent Application Publication
(10) Pub. No.: US 2008/0081979 A1
(43) Pub. Date: Apr. 3, 2008

(54) Medical diagnostic system data exchange method and system

(75) Inventor: Jeffrey Dale Solliday-McRoy, Menomonee Falls, WI (US)

Correspondence Address: Patrick S. Yoder;FLETCHER YODER, P.O. Box 692289, Houston, TX, 77269-2289, US

(73) Assignee: General Electric Company

(21) Appl. No.: 11/521,865

(22) Filed: Sep. 15, 2006

(51) Int. Cl. A61B 5/05

(52) U.S. Cl. 600/407

(57) ABSTRACT

A technique is disclosed for exchanging data between a medical diagnostic imaging system and a remote service provider. The system includes the use of an instant messaging (IM) agent stored an operative on the imaging system and at the remote service provider. A human operator at the imaging system may initiate an IM session with the service provider, or such sessions may be initiated either by the service provider or automatically by the imaging system or by computers at the service provider. The IM session may include the exchange of service data, log files, error files, as well as general information on the operation and servicing of the imaging system. The IM session may supplement or replace telephonic exchanges, electronic messages, or even certain personal visits by service engineers.

### BACKGROUND

The present invention relates generally to the field of medical diagnostic systems. More particularly, the invention relates to a technique for communicating information to and from such systems via an instant messaging agent.

Over recent years medical diagnostic imaging equipment has evolved to provide improved connectivity to service providers, remote systems, hospital and clinic networks, and so forth. For example, many fixed medical diagnostic imaging systems were at one time equipped with communications circuitry that permitted them to be contacted from a service provider so as to detect or extract data regarding operation of the system. In many cases, the system operators were themselves unaware that such contacts were being made, with service providers ensuring the good operating condition of the systems with minimal intervention by the operators. Later evolution in such connectivity solutions included the provision of browsers and similar interfaces on imaging systems themselves. A scanner operator could then contact a service provider by formulating an electronic message (i.e., email) that would serve as a service call for attention by the remote service provider. The service provider could respond by a similar electronic communication, intervene to extract log and error files, load software onto the system, and so forth. Such solutions proved enormously useful in improving the responsiveness and quality of operational service to the imaging systems. At present, such solutions are available on a range of imaging systems, including ultrasound systems, magnetic resonance imaging (MRI) systems, X-ray and computed tomography (CT) systems, positron emission tomography (PET) systems, and so forth.

Further improvement in connectivity solutions for medical diagnostic imaging systems is still needed. For example, existing solutions do not provide an immediate response mechanism that can address pressing needs at the systems. Rather, they generally serve to submit a service request which enters a service queue to be addressed in an appropriate order. Similarly, while many problems could be addressed immediately by a service engineer or a field engineer, there is not at present a mechanism on the systems to contact the appropriate personnel for the immediate service needs. Further, while electronic mail submission of service requests has presented the potential for an operator-free communication, the field has not yet moved completely to a solution which would allow a system to contact a service provider independent of an operator-initiated message for immediate exchange of necessary log and error files.

There is a need, therefore, for an improved connectivity solution that permits the immediate exchange of information between a remote service provider and a medical diagnostic imaging system.

### BRIEF DESCRIPTION

The invention provides a novel approach to communication exchanges with medical diagnostic systems designed to respond to such needs. The technique makes use of an instant messaging agent which is loaded on and active on the medical diagnostic imaging system. The technique may be used on any suitable system, including ultrasound systems, MRI systems, X-ray and CT systems, PET systems, and so forth. The instant messaging agent may initiate exchanges with the remote service provider generally or with specific persons at a service provider for addressing service needs in an immediate fashion. The persons or providers may be stored in a contact list on the imaging system, with similar contact lists being stored at the service provider, or on computers utilized by specific service personnel.

Data exchanges between the imaging systems and the service provider or personnel may be initiated by an operator at the imaging system, or automatically by the imaging system. In either event, specific service providers or persons may be contacted by an instant message sent from the system. Because the system is capable of announcing its presence (e.g., it operative state) and is aware of the status of service providers and persons, immediately addressing of the service needs is afforded.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical overview of a medical diagnostic imaging system equipped with and instant messaging (IM) agent for exchanging service information and other data with a remote service provider;

FIG. 2 is diagrammatical representation of a series of imaging systems in a medical institution equipped with IM agents for similarly exchanging information with remote service providers;

FIG. 3 is a similar diagrammatical representation of a series of medical diagnostic imaging systems in an institution, but wherein a communications manager in the institution is provided for instant messaging exchanges with a remote service provider where certain systems in the institution are not provided with such agents;

FIG. 4 is a representation of a partial instant messaging contact list as it might appear on one of the medical diagnostic imaging systems illustrated in the previous figures for contacting and exchanging information with a remote service provider in accordance with aspects of the invention;

FIG. 5 is an exemplary “session page” illustrating exchanges between a medical diagnostic imaging system equipped with an IM agent and a remote service provider to provide operational service to the imaging system; and

FIG. 6 is a flow chart illustrating exemplary logic in initiation and exchange of service data between an IM-equipped medical diagnostic imaging system and a remote service provider in accordance with aspects of the invention.

### DETAILED DESCRIPTION

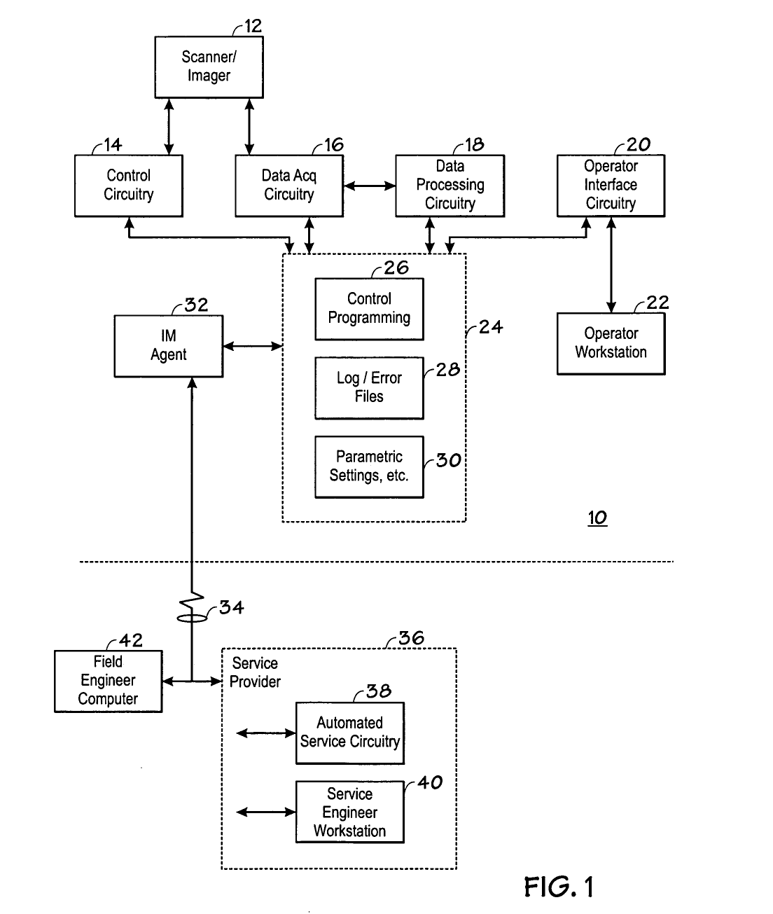

Turning now to the drawings, and referring first to FIG. 1, an IM-equipped medical diagnostic system data exchange system is illustrated and designated generally by the reference numeral 10. In the illustrated embodiment, the system includes a medical diagnostic imaging system, sometimes referred to as a scanner or imager 12. As will be appreciated by those skilled in the art, the scanner/imager 12 may include any suitable modality system, such as ultrasound systems, MRI systems, CT and X-ray systems, PET imaging systems, and so forth. The scanner/imager will generally be capable of creating image data of a subject of interest based upon the physics of its particular modality. The imager/scanner 12 operates under the control of control circuitry 14 which will typically initiate scanning sequences, implement particular scanning protocols, and regulate the acquisition of image data that will be digitized for reconstruction of useful images. Data acquisition circuitry 16, then, receives such digitized data and stores the data for further processing, enhancement, and reconstruction into the ultimate useful images.

The data acquisition circuitry will typically operate, again, in coordination with the particular physics of the scanner/imager 12. By way of example, for magnetic resonance imaging, the acquisition circuitry will receive digitized information resulting from detection of radiofrequency echoes returned from a subject of interest to fill K-space lines. In CT imaging and X-ray imaging, on the other hand, the acquisition circuitry will typically received a stream of digitized data representative of intensities of X-ray radiation received at pixel locations of a digital detector. Ultimately, the data acquisition circuitry 16 transmits the received data to data processing circuitry 18. Again, depending on the form of the data and the physical parameters that it represents, the data processing circuitry 18 will at least partially process the data by appropriate filtering, dynamic range adjustments, noise reduction, and so forth. The data processing circuitry 18 may also reconstruct images for display. In general, such reconstruction will be based upon the physics of the scanner/imager 12, and may include computation of pixel or voxel data by 2D fast Fourier transforms (e.g., for MR imaging), reconstruction of slice images from projection data (e.g., in CT images), and so forth.

System 10 also includes operation interface circuitry 20. The operator interface circuitry will allow an operator to both initiate imaging sequences, as well as to adjust the parameters of the imaging system. The operator interface circuitry 20 may also display images as they are reconstructed from the acquired data. The operator interface circuitry will typically communicate with an operator workstation 22, which will include a monitor, input and output devices, such as keyboards, mice, printers, and so forth.

The operator interface circuitry 20, as well as the other system circuitry will typically communicate with memory 24 that stores data and programs needed to operate the scanner/imager 12. In the illustrated embodiment, such memory may include control programming 28, such as imaging protocols, pulse sequence descriptions, data acquisition circuitry control routines, and so forth. The memory will also typically store log and error files as indicated at reference numeral 28. Such files may be created during operation of the system, to record both normal operating histories, as well as abnormal histories and events as these are detected by various sensors or control programs of the scanner/imager, the control circuitry, data acquisition circuitry or any other circuitry of the system. Finally, the memory 24 will typically store parametric settings, such as the settings implemented by the control programming in regulating operation of the scanner/imager. These may include, for example, position settings for the various system components, timing settings, calibration settings, and so forth.

As will be appreciated by those skilled in the art, in normal operation, the control circuitry 14 will draw upon information stored in the memory 24 for operation of the scanner/imager 12, and acquisition and processing of data based upon inputs from an operator via the operator workstation 22. In certain systems, the operator control may also include input via hand-held devices, buttons or controls directly at the scanner/imager, and so forth. From time to time, as new programs, routines, settings and so forth are stored on the system, these will be included in and possibly replace information in the memory 24. Moreover, during abnormal operation of the system, error files and log files may be created and stored in the memory, as well as “snap shots” representing settings and operating conditions of the system immediately preceding, during and, where appropriate, after abnormal events are detected. As noted below, such events and files may assist in diagnosing possible serviceable events.

In the illustrated embodiment, an IM agent 32 is also stored on the system, and may include data stored in the memory 24 or data and routines stored separately. As will be appreciated by those skilled in the art, any IM protocol used by commercially available IM software (i.e., clients) may be employed for this purpose. IM clients are available from a number of software manufacturers and distributors. By way of example, the invention has been implemented by a use of an IM agent using the same protocol as an IM client package available under the commercial designation “Sametime”, from Lotus software a division of International Business Machines a Corporation of Armonk, N.Y. Alternatively, the IM agent may use a custom or proprietary protocol. It should be noted, however, that an “IM agent” in the present context differs substantially from an “IM client” as that term is commonly used in the art. Whereas an IM client is a graphical user interface based application provided for human interfacing via the IM protocol, an IM agent is an automated application, or robot, that can communicate with humans or automated services. In the present context, the IM agent runs on the imaging system and can provide greatly enhanced functionality, as described below. The agent may also provide IM client functionality, enabling an operator at the imaging system to communicate with a remote service provider for operational servicing of the system. Where desired, such client functionality may be more limited than that typically provided by IM client software, such as by restricting use or contacts of the client functionality to those needed for operational servicing of the system by a specific service provider (i.e., the provider contracted to maintain the system in good working order).

The IM agent software may be delivered with the system 10 or may be added or retrofit to the system after initial installation. Moreover, the IM agent 32 may exist and run in parallel with other service software, such as software that allows for electronic messaging to remote service providers, access by remote service providers to data stored in memory 24, and so forth. In general, the IM agent will likely be customized, and in some cases, highly customized to interface with the functionality of the imaging system. In many cases it may be desirable to adapt the agent for specific modality systems (e.g., CT, MRI, PET, ultrasound, etc.) and even for specific system models or configurations. Such customization may greatly enhance the ability of the agent to perform such functions as addressing data content stored on the system, locating such data and data files, launching routines added to or existing on the systems, accessing error logs or maintenance records, and so forth. Where service software and mechanisms (e.g., operational diagnostics routines) are stored on the systems, the agent may automatically interface with such software, such as for diagnosis of serviceable problems. As discussed below, remote service providers may even launch such routines via interaction with the IM agent to provide rapid and targeted evaluation of serviceable conditions.

As will be appreciated by those skilled in the art, the IM agent 32 will typically include software tools that provide both awareness and real-time collaboration features to enable an interactive, text-based interface to data stores and databases both on the system 10, and remote from the system. The agent allows for such awareness, in addition to presence detection. Again, as will be appreciated by those skilled in the art, “presence” in the present context may be defined as the ability for the user or system 10 to express its current state of activity to others on a network. Presence may be used to inform remote service providers or others whether a particular user or system is available and able to respond to messages. In certain IM program environments, such presence may include states such as “Online-Available”, “Online-Away”, “Online-Do Not Disturb”, and “Offline”. This, however, is not intended to be an exhaustive list of the various states which may be communicated by the presence function of the IM agent.

“Awareness” may be defined, in the present context, as the ability to sense the presence of others in the IM environment. As with presence, awareness allows the initiator or an IM session to know or feel confident of the likelihood of receiving an immediate response when initiating a session. Awareness also allows the initiator to make decisions about the medium to use when conducting sessions.

In the present implementation, the presence and awareness features can be used to initiate exchanges between the system 10 and a remote service provider. As illustrated in FIG. 1, for example, system 10 is coupled to an external network via a network link 34. The network link 34 may include any suitable type of connection, such as a wide area network, wireless network, cellular network, and so forth. In a present implementation, depending upon the level of security available and required, network 34 may include the Internet. The service provider, as indicated by reference numeral 36, is linked to the system 10 via the network. The service provider will typically include automated service circuitry 38 as well as service engineer workstations 40. The service provider 36 may be relatively local to the system 10 or may be completely remote from the system. That is, the service provider may contract with a medical institution in which system 10 is situated and provide remote service for assuring the proper operation of system 10 via the IM agent 32, network 34, and the automated service circuitry 38.

The automated service circuitry 38 may be resident on application-specific or of general purpose computers at the service provider 36. In general, such circuitry may be programmed to remain available through the presence and awareness functions of the IM environment to receive messages from the IM agent 32 and even to automatically respond to the operator of system 10 or to the system 10 operating autonomously. The automated service circuitry 38 may, as described in greater detail below, acknowledge the receipt of instant messages received from system 10, return information and acknowledgments, provide data, software, programming instructions or extract files, such as log and error files via the IM agent. The service engineer workstation 40 may be provided with similar software, including its own IM agent (not represented in FIG. 1). As service engineer at the workstation, then, may communicate with system 10 in real-time via the IM agent 32. Similarly, to the automated service circuitry 38, a service engineer operating at workstation 40 may, then, exchange messages with an operator at system 10 or with the system operating in an automated fashion. The service engineer workstation may, for example, log onto system 10, where screening or access control routines are provided, view log and error files, view parametric settings, view control programming, or any other information available for rendering service to the system.

In the illustrated embodiment, the service provider may also make use of field engineer computers as represented generally at reference numeral 42. Such computers may include field engineer laptops, palm computers, or any other circuitry configured to communicate with the IM agent 32 via network 34. The field engineer computer 42 may be provided with automated service circuitry similar to that represented at reference numeral 38, as well as with circuitry and programming similar or identical to that provided for the service engineer workstation 40. The field engineer may thus address immediate issues with the imaging system 10 by means of the network 34 and through instant messaging exchanges enabled by the IM agent 32 on system 10. In certain embodiments, the field engineer may not require the functionality of a special IM agent such as that provided on serviced imaging systems. Indeed, this may generally be the case for many field engineers. In such cases, the field engineer may be provided with an IM client only, so that exchanges can be made with imaging system IM agents, and with operators at the imaging systems via the client functionality of the IM agents.

It should be noted that, as used herein the term “service” or “operational service” generally refers to the provision of information, data, and so forth from the remote service provider to the system 10 to ensure the proper operation of serviced systems (e.g., maintenance, upgrading, periodic surveying of proper operation, etc.), as well as the evaluation of the operational state of the system by the service provider, such as via examination of log and error files, parametric settings, control routines, and so forth. In the medical diagnostics field, teleradiology or telemedicine is sometimes referred to as service, although such techniques are not the focus of the present invention.

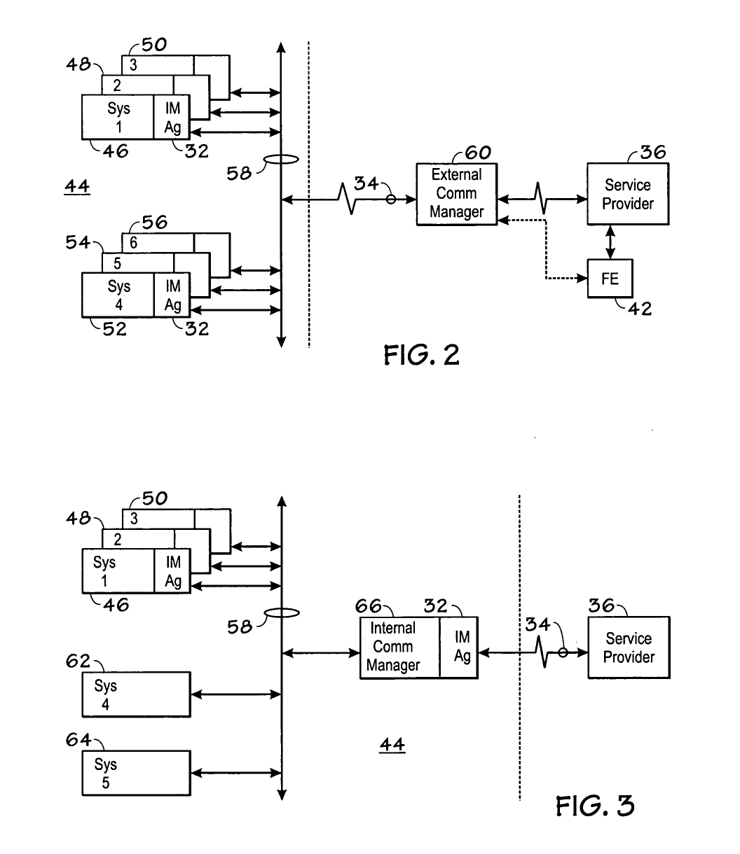

Several possible system topologies may be envisaged based upon the overview summarized in FIG. 1. Two such topologies are generally illustrated in FIGS. 2 and 3. In the topology of FIG. 2, several medical diagnostic imaging systems are situated in an institution generally represented by reference numeral 44. The institution may be in a single location, or in multiple locations. Moreover, the institution may include imaging systems in a single office, floor, department, and so forth, or the imaging systems may be included in several such departments, floors or buildings. The imaging systems, identified in FIG. 2 generally by reference numerals 46-56, will each include circuitry such as that described above with reference to FIG. 1, as well as its own IM agent 32. Each system may be equipped to communicate over a local area network (LAN) 54 within the institution. Via the LAN, then, communications may be channeled through network 34 as described above. In certain implementations one or more of the systems may communicate separately from the LAN. In the topology of FIG. 2, moreover, an external communications manager 60 may oversee, filter, or otherwise control communications between institution 44 and the service provider 36 or field engineer 42. In general, the communications manager 60 may implement additional firewalls which may supplement firewalls within the institution 44. The communications manager 60 may also limit access to certain information from the institution, such as images and other patient records that may include patient data. Such management may be one-way or two-way. That is, the communications manager 60 may allow the imaging systems within the institution to convey certain information or transmit certain types of files to the remote service provider, but may preclude the service provider from accessing certain types of information from the imaging systems. In general, log files, error files, parametric settings and so forth may be provided to the service provider under certain contractual or confidentiality arrangements. Other information, such as information that may identify particular patients, may not be accessible at all to the remote service provider.

FIG. 3 represents a different topology in which certain imaging systems 62 and 64 are not provided with or cannot support an instant messaging agent of their own. In this topology, the communications manager 66 may be provided internal to the institution 44. The communications manager is illustrated as being coupled to the LAN 58, and can communicate both with systems 46, 48 and 50, as well as with systems 62 and 64. In this topology, the internal communications manager 66 has its own instant messaging agent 32. The instant messaging agent 32 may, then, be used to communicate with the service provider 36 via the network 34. Where information is to be gathered from files stored in imaging systems 62 and 64, then, the internal communications manager 66 may access certain information and transfer it to the remote service provider upon demand, such as during an IM session. In practice, the internal communications manager 66 may be part of a management workstation, such as in a radiology department. It should also be noted that an internal communications manager 66 may be used in conjunction with an external communications manager 60 of the type illustrated in FIG. 2. Also, the internal communications manager 66 may serve certain or all of the functions of the external communications manager 60, such as requiring authentication of service providers, filtering certain information, denying access to certain types of data, and so forth.

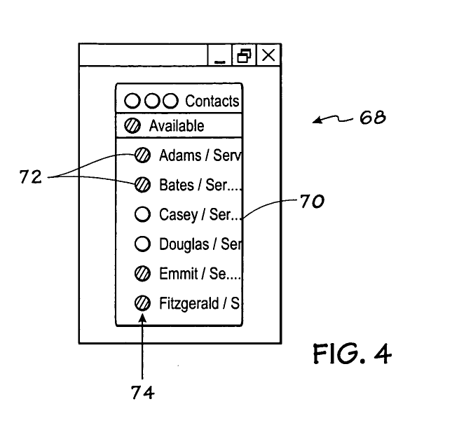

As will be appreciated by those skilled in the art, the IM agent and IM environment provide user viewable pages or screens, including screens listing contacts, and so forth. A portion of an exemplary interface screen 68 is illustrated in FIG. 4. Again, the screen 68 may be a portion of a display on a conventional monitor, such as a monitor operating on the operator workstation 22, or service engineer workstation 40, or even the field engineer computer 42, all shown in FIG. 1. The interface screen 68 displays a contact window 70 which may list a series of contacts and service providers. The individual contacts may include, for example, individual specialized service engineers at the service provider capable of addressing service needs in real-time as they arise at the diagnostic imaging system. Such contacts may also include, for example, field engineers that can be contacted by wireless communication, as well as field engineer laptops, and so forth. Still further, the contact lists may include other IM agents operative on other imagines systems. Based upon the presence and awareness functions of the IM environment, the contacts window 70 will typically include lists of the contacts, as indicated by the alphabetized list shown in FIG. 4, as well as some sort of status indicator 74. In the illustration of FIG. 4, for example, the status indicators shown in hatched or gray will indicate that the listed persons or institutions are present and available, and that an IM session may be initiated with them. The list itself provides the awareness function of the IM environment.

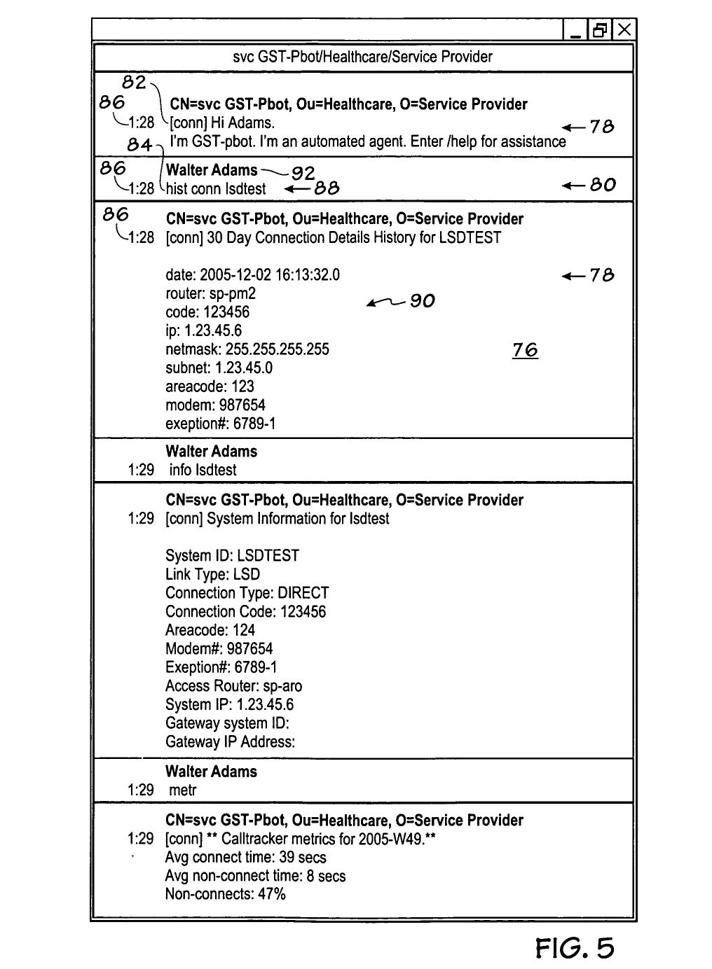

Based upon the list of available contacts, then, the system 10 illustrated in FIG. 1, or an operator at the system, or even, in an opposite sense, a service engineer or field engineer, may contact one another to initiate an IM session. An exemplary data exchange in such an IM session is illustrated generally in FIG. 5. While it should be understood that such sessions may include intuitive English or other language exchanges, certain codes, images, file descriptions, file names, and so forth may be exchanged in a typical session. The session page, designated generally by reference numeral 76 in FIG. 5 may include a series of messages exchanged between the system 10, or its operator, as well as between the remote service provider or a human service technician or engineer at the service provider. A typical session will include a series of messages 78 and may include exchange of system information as designated generally at reference numeral 80. In the illustrated embodiment, an automatic message 82 has been generated to initiate the IM session. In actual practice, certain of the data illustrated in FIG. 5 may be hidden from a human user to facilitate data exchange and to make the exchange more intuitive. In the exchange illustrated in FIG. 5, a system has initiated data exchange with an IM partner Walter Adams. The partner, in this case, is a service engineer who initiates a test of the system via a message 84. Message 84, in this case, is a reply to the system. Each message exchanged includes a time stamp 86 to permit reconstruction of the exchange and understanding of the sequence of messages that were exchanged between the service provider and the system.

In the illustrated embodiment, the service engineer Walter Adams is communicating with the system to perform an analysis called “hist conn lsdtest”, as indicated by the message text 88. In response, the system has automatically returned communications settings and data 90 identifying, for example, a date, a router, an IP address, network identifications, and so forth. Moreover, in the illustrated embodiment, Walter Adams, as indicated by reference numeral 92 is the human partner, whereas on the system side the partner is an automated collection and data exchange robot. As noted above, such instant messaging data exchanges may occur between human operators on both sides, between humans and automated system software or between the automated systems themselves. However, it is presently contemplated that the invention provides a substantial advantage and facility in enabling human system operators to exchange data with human service providers, such as to ask questions, request information on the operation of the systems, identify problems occurring on systems, access and send snapshots, error files, log files, and so forth. The invention also provides unprecedented utility in enabling imaging systems to send urgent messages to maintenance and service providers, both machine and human. As will be appreciated by those skilled in the art, medical diagnostic imaging systems, in particular, may encounter many different problems (e.g., quenching of a superconducting magnet in an MRI system) that may benefit from very rapid notification and response of a service provider.

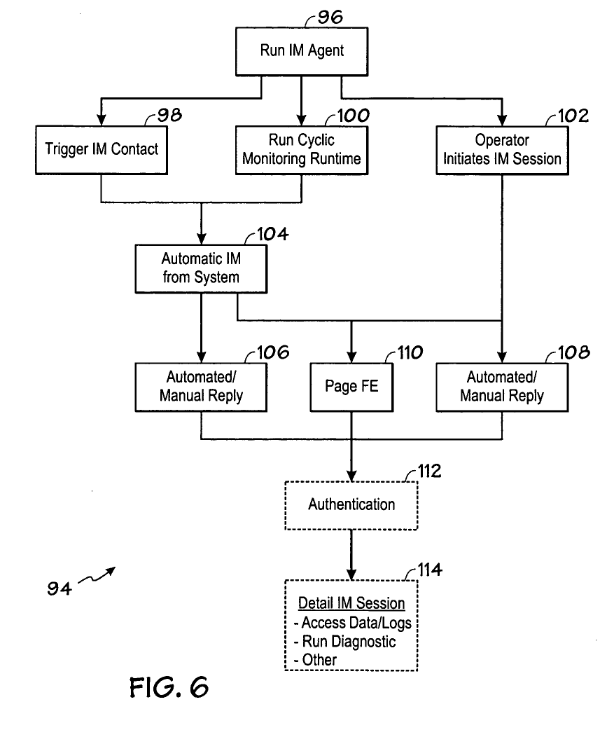

FIG. 6 illustrates exemplary steps in initiating and exchanging information during an IM session via an IM agent operating on a medical diagnostic imaging system. The process, indicated generally by reference numeral 94, begins with initiating and running the IM agent as indicated at step 96. The IM agent may initiate sessions in several ways. For example, the IM agent may trigger an IM contact, such as upon the occurrence of an event on the system. Such events may include errors occurring, or data or parameters detected on the system falling outside of anticipated ranges, and so forth. Alternatively, the IM agent may initiate a session by means of a cyclic monitoring routine as indicated at reference numeral 100. Such routines may initiate sessions cyclically or at predetermined integrals or based upon a schedule. Such sessions may be initiated, for example, for routine exchange of data with a service provider, such as on a daily, weekly or monthly basis. Alternatively, the session may be initiated by an operator as indicated at reference numeral 102. Such a session will typically be initiated by an operator clicking a mouse with a cursor located over a contact indicated as available in a display such as that illustrated in FIG. 4 above. Thereafter, the session page will be displayed as indicated in FIG. 5.

When an automated session is initiated, such as by steps 98 and 100, an automatic instant message is sent form the system to the remote service provider as indicated at step 104. The automated message may prompt an automated reply as indicated by reference numeral 106. In such a case, automated routines operating on both sides of the exchange may receive and send messages, such as to prompt the access and transmission of certain types of information and files, and so forth. The message 106 may also be a manual message that is input by a human operator either at the service provider, or via a field engineer's computer or workstation.

A similar automated or manual reply 108 may result from an operator-initiated IM session 102. Where possible, it may be preferable for a human service provider to respond to the IM message from the system. However, where such a response is not desired or it is not possible, an automated reply may be sent to the human operator at the system, such as indicating that the message was received, or accessing and providing the operator with configuration information, system data, as well as pages for inputting descriptions of problems or service needed. Other replies may include pages of a field engineer or other service technician as indicated at reference numeral 110. Such pages may occur by use of any suitable communications hardware, software and protocols, including conventional cellular telephones.

Following initiation of the session, any number of information exchange and information exchange control steps may follow. In the session illustrated in FIG. 6, for example, an authentication step 112 may follow. Such a step may be required, for example, to authenticate that the person initiating the exchange is authorized to do so, such as by reference to contractual relationships and so forth. Such authentication may also be used, such as by a communications management component to ensure that the service provider is authorized to access particular data, files, logs and so forth on the imaging system. Such authentication may include, for example, user name and password inputs. Reference numeral 114 refers generally to an ongoing detailed IM session in which messages may be exchanged, data and log files may be accessed, diagnostic routines stored on the imaging system or remote from the imaging system may be run, and so forth. Following the session, a log may be made of the exchange, and the session page may be closed.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A system for exchanging data with a medical diagnostic imager comprising:
an imager for acquiring image data of a subject of interest that can be processed to provide a diagnostic image; and
an instant messaging agent stored and operative on the system for initiating instant messaging data exchanges between the system and a remote service provider for operational servicing of the imager.

2. The system of claim 1, comprising an operator interface for providing on the imager a list of service provider contacts with whom instant messaging data exchange sessions may be initiated.

3. The system of claim 2, wherein the list includes indicia of whether a contact is currently available for an instant messaging session.

4. The system of claim 1, comprising memory for storing error files, log files and parametric settings for the imager, and wherein the instant messaging agent can access the error files, log files and parametric settings for transmission to the remote service provider during an instant messaging session.

5. The system of claim 1, wherein the instant messaging agent is configured to initiate instant messaging sessions without operator intervention at the system.

6. The system of claim 1, wherein the instant messaging agent is provided on a management workstation coupled to a plurality of imagers that do not have instant messaging agents.

7. The system of claim 1, comprising a communications manager that at least partially controls data exchanged between the system and the remote service provider during an instant messaging session.

8. The system of claim 7, wherein the communications manager prevents files or data stored on the system from being accessed by the remote service provider.

9. A system for exchanging data with medical diagnostic imaging systems comprising:
a plurality of medical diagnostic imaging systems each including an imager for acquiring image data of a subject of interest that can be processed to provide a diagnostic image; and
a plurality of instant messaging agents, an instant messaging agent being stored and operative on each imaging system for initiating instant messaging data exchanges between the respective system and a remote service provider for operational servicing of the respective imager.

10. The system of claim 9, wherein the imaging systems include imagers of at least two different imaging modalities.

11. The system of claim 9, comprising an internal network coupled to the diagnostic systems, the instant messaging agents exchanging data between the respective imaging systems and the remote service provider via the internal network.

12. The system of claim 11, comprising a plurality of imaging systems that do not have instant messaging agents, and a communications management station coupled to the imaging systems via the internal network for engaging in instant messaging sessions with the remote service provider and for accessing data from the imaging systems during such sessions for operational servicing of the imaging systems.

13. A system for exchanging data with medical diagnostic imaging systems comprising:
a first set of medical diagnostic imaging systems of different imaging modalities, each imaging system of the first set including an imager for acquiring image data of a subject of interest that can be processed to provide a diagnostic image, each imaging system of the first set further including a respective instant messaging agent for initiating instant messaging data exchanges between the respective system and a remote service provider for operational servicing of the respective imager;
a second set of medical diagnostic imaging systems not having an instant messaging agent;
a local area network coupled to at least the imaging systems of the second set; and
a communications management station coupled to at least the imaging systems of the second set via the local area network for engaging in instant messaging sessions with the remote service provider and for accessing data from the imaging systems of the second set during such sessions for operational servicing of the imaging systems of the second set.

14. The system of claim 13, wherein the imaging systems of the first set are coupled to the local area network and exchange data with the remote service provider during instant messaging sessions via the local area network.

15. A method for providing operational servicing to a medical diagnostic system comprising:
initiating an instant messaging session between the medical diagnostic system and a remote service provider via an instant messaging agent stored and operative on the medical diagnostic system; and
exchanging service data between the medical diagnostic system and the remote service provider during the instant messaging session.

16. The method of claim 15, wherein the medical diagnostic system includes an imager configured for acquiring image data of a subject of interest that can be processed to provide a diagnostic image.

17. The method of claim 15, comprising transmitting at least one file from the medical diagnostic system to the remote service provider via the instant messaging agent for evaluation of an operational state of the medical diagnostic system.

18. The method of claim 17, comprising limiting access by the remote service provider to files and data stored on the medical diagnostic system.

19. The method of claim 15, wherein the instant messaging session is automatically initiated by the medical diagnostic system without operator intervention.

20. The method of claim 15, wherein the instant messaging session is initiated by the remote service provider.
